(19) United States
(12) Patent Application Publication
(10) Pub. No.: US 2011/0038957 A1
(43) Pub. Date: Feb. 17, 2011

(54) NOVEL NUTRACEUTICAL COMPOSITIONS CONTAINING STEVIA EXTRACT OR STEVIA EXTRACT CONSTITUENTS AND USES THEREOF

(76) Inventors: Ann Fowler, Rheinfelden (CH); Regina Goralczyk, Grenzach-Wyhlen (DE); Claus Kilpert, Mannheim (DE); Annis Olivia Mayne-Mechan, Moehlin (CH); Bernd Mussler, Lahr (DE); Adrian Wyss, Basel (CH)

Correspondence Address: NIXON & VANDERHYE, PC, 901 NORTH GLEBE ROAD, 11TH FLOOR, ARLINGTON, VA, 22203, US

(21) Appl. No.: 12/745,421

(22) PCT Filed: Dec. 3, 2008
(86) PCT No.: PCT/EP2008/010231

§ 371 Date: Oct. 29, 2010

(30) Foreign Application Priority Data
Dec. 3, 2007 (EP) 07023334.1

(51) Int. Cl. A61K 31/19; A61K 36/28; A61P 25/00

(52) U.S. Cl. 424/725; 514/572

(57) ABSTRACT

The invention relates to a novel nutraceutical composition containing Stevia extract or its constituents, such as steviol and stevioside, as active ingredient(s). The term “nutraceutical” as used herein denotes usefulness in nutritional, pharmaceutical and veterinary fields of application. The compositions are useful for improvement of cognitive functions, such as learning, memory and alertness, and psychotic stability.

### FIELD OF THE INVENTION

The present invention relates to a novel nutraceutical composition or food additive comprising Stevia extract or its constituents, such as steviol and stevioside, as active ingredient(s) to improve cognitive functions, such as learning, memory and alertness, as well as relieving psychosocial pressure.

### BACKGROUND OF THE INVENTION

Memory, learning and alertness rely on neuronal circuits in the midbrain, especially in the hippocampus where information is processed and memory is consolidated. Mental performance and learning are dependent on synaptic plasticity; i.e. strengthening of neuronal connections by the recruitment of new receptors, formation of new synapses and eventually the generation of new neuronal connections.

The formation of (long-term) memory and the efficient functioning of the brain depend on synthesis of new proteins for the reinforcement of communicative strength between neurones. The production of new proteins devoted to synapse reinforcement is triggered by chemical and electrical signals within neurones.

Long term potentiation (LTP) is the term used to describe the long-lasting enhancement of synaptic transmission (hours in vitro, days or weeks in vivo) which occurs at particular synapses within the central nervous system (CNS) following a short, conditioning, burst of presynaptic electrical stimulation (approximately 100 Hz for 1 second). This phenomenon is widely considered to be one of the major mechanisms by which memories are formed and stored in the brain. LTP has been observed both in vitro and in living animals. Under experimental conditions, applying a series of short, high-frequency electrical stimuli to a synapse can potentiate the strength of the chemical synapse for minutes to hours. Most importantly, LTP contributes to synaptic plasticity in living animals, providing the foundation for a highly adaptable nervous system.

Two different receptor types are primarily involved in the process of LTP, namely the N-methyl-D-aspartate (NMDA) receptor complex and the α-amino-3-hydroxy-5-methyl-4-isoxazole propionic acid (AMPA) receptor. During LTP, the major excitatory neurotransmitter, glutamate, is released from the presynaptic neurone, binds to and activates the AMPA receptor on the postsynaptic membrane, leading to its depolarisation. At resting membrane potentials, the NMDA receptor channel is blocked by magnesium ions, but depolarisation of the postsynaptic membrane removes this block, enabling NMDA receptor activation and subsequent entry of calcium into the cell. This rise in intracellular calcium is believed to activate protein kinases, leading to gene transcription and the construction of reinforcing proteins (Niehoff (2005), The Language of Life: How Cells Communicate in Health and Disease, 210-223) and resulting in enhanced sensitivity of the AMPA receptor, thus further facilitating neurotransmission and maintenance of LTP.

NMDA receptors are composed of assemblies of NR1- and NR2-subunits; the glutamate binding domain is formed at the junction of these subunits. In addition to glutamate, the NMDA receptor requires a co-agonist, glycine, in order to modulate receptor function. The glycine binding site is found on the NR1 subunit, while the NR2 subunit possesses a binding site for polyamines, regulatory molecules that modulate the functioning of the NMDA receptor.

The amino acid glycine is thus known to act as a positive allosteric modulator and obligatory co-agonist with glutamate at the NMDA receptor complex (Danysz and Parsons 1998 Pharmacol. Rev., 50(4):597-664). Glycine transporters (GlyT) play an important role in the termination of postsynaptic glycinergic actions and maintenance of low extracellular glycine concentrations by reuptake of glycine into presynaptic nerve terminals or glial cells. The termination of the action of glycine is therefore largely mediated by rapid reuptake. Two glycine transporters, GlyT1 and GlyT2, are known and are characterised by 12 putative transmembrane regions, while three variants of GlyT1 (GlyT1a, b, and c) encoded from the same gene have been identified (Borowsky and Hoffman 1998 J. Biol. Chem., 273(44):29077-29085).

GlyT1 is the only sodium chloride-dependent glycine transporter in the forebrain, where it is co-expressed with the NMDA receptor. At this site, GlyT1 is thought to be responsible for controlling extracellular levels of glycine at the synapse (López-Corcuera et al 2001 Mol. Membr. Biol., 18(1):13-20), resulting in modulation of NMDA receptor function.

Indeed, in the presence of the selective GlyT1 antagonist N-[3-(4′-fluorophenyl)-3-(4′-phenylphenoxy)] propylsarcosine (NFPS), enhanced NMDA receptor responses in CA1 pyramidal cells were observed upon Schaffer collateral stimulation in both mouse and rat hippocampal slice preparations (Bergeron, et al 1998, Proc. Natl. Acad. Sci. USA, 95(26): 15730-15734). In vivo, systemic administration of NFPS increased LTP in the dentate gyrus and enhanced prepulse inhibition of the acoustic startle response in adult mice, indicating that inhibition of GlyT1 affects NMDA receptor function in a behaviourally-relevant way (Kinney, et al 2003, J. Neurosci., 23(20):7586-7591).

Such data highlight the potential usefulness of compounds which can enhance NMDA receptor synaptic function by elevating extracellular levels of glycine in the local microenvironment of synaptic NMDA receptors, for the prevention of psychotic syndromes and for maintaining or boosting physiological cognitive functions, such as memory and learning, in normal individuals.

There is an increasing interest in the development of compounds, as well as nutraceutical compositions, that may be used to improve learning, memory and alertness, in both elderly and young people, individuals who need especially high memory and attention in their daily work, including students, construction workers, drivers, pilots, physicians, salespeople, executives, housewives, “high performance professionals” and people who are under mental or daily stress as well as persons who are prone to psychiatric instability, such as schizophrenia.

Thus, a compound or nutraceutical composition which enhances NMDA receptor function and enables improvements in learning, memory and alertness would be highly desirable.

Stevia extracts have been added to beverages/food compositions in the past, (for example see Japanese Kokai 2005-278467 to Nippon Paper Chemical Co., Ltd), but the Stevia extract functioned as a sweetener.

### DETAILED DESCRIPTION OF THE INVENTION

It has been found, in accordance with this invention that compounds of Formulae I and II, below, are activators of hippocampal function, through their ability to induce LTP, and as such are useful as nutraceuticals and/or pharmaceuticals which enhance cognitive functions. These compounds can function either by inhibiting the glycine transporter, GlyT1, thus inhibiting reuptake of glycine, or by activation of another pathway which leads to LTP induction, or by both mechanisms. Thus, this invention relates to a method of enhancing cognitive functions by administering a cognitive-function enhancing amount of a compound of Formula I, II, or mixtures thereof to an animal (including humans).

It has also been found in accordance with the invention that Stevia extract has the ability to inhibit glycine reuptake by inhibiting the glycine transporter, GlyT1. The resulting increase in extracellular glycine levels leads to enhanced activation of NMDA receptors, which is the first step towards inducing transcriptional activation of a number of genes and subsequently to induce LTP, the main cellular mechanism involved in memory formation and consolidation.

Additionally, it has been found in accordance with this invention that steviol, isosteviol and stevioside, compounds which can be found in Stevia extracts, induce LTP through a different mechanism. As both processes have the same biological benefits, i.e. they both facilitate hippocampal function, another aspect of this invention is the use of one or more of these active ingredients to enhance cognitive functions.

Therefore one aspect of the invention is a novel nutraceutical composition, comprising Stevia extract or one or more compounds of Formula I or II to enhance cognitive function. Particularly preferred compounds of Table 1 are steviol and stevioside, in addition to isosteviol.

Formula I: See Table 1 for details of R groups

TABLE 1

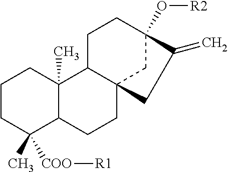

Structures of steviol and related glycosides (Formula I). Glc, Xyl
and Rha represent, respectively, glucose, xylose and rhamnose
sugar moieties (Kuznesof (2007)
Steviol glycosides - chemical and technical assessment,
68^{th} JECFA meeting).

| Compound name | R1 | R2 |
|---|---|---|
| Steviol | H | H |
| Steviolbioside | H | β-Glc-β-Glc (2 → 1) |
| Stevioside | β-Glc | β-Glc-β-Glc (2 → 1) |
| Rebaudioside A | β-Glc | 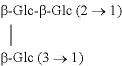 |
| Rebaudioside B | H | 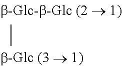 |
| Rebaudioside C (dulcoside B) | β-Glc | 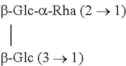 |
| Rebaudioside D | β-Glc-β-Glc (2 → 1) | 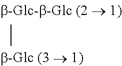 |
| Rebaudioside E | β-Glc-β-Glc (2→ 1) | β-Glc-β-Glc (2 → 1) |
| Rebaudioside F | β-Glc | 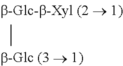 |
| Rubusoside | β-Glc | β-Glc |
| dulcoside A | β-Glc | β-Glc-α-Rha (2 → 1) |

Formula II: Isosteviol (CAS number, 27975-19-5) is a decomposition product of steviol glycosides.

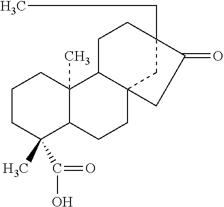

The compounds of Formula I and II can either be synthetically produced using known methods, be extracted from natural sources, such as plants, using known extraction procedures, or they may be used as a component of a plant extract, preferably a Stevia extract, which contains sufficient amounts of steviol and/or stevioside to be an effective enhancer of hippocampal function.

### BRIEF DESCRIPTION OF THE FIGURES

FIG. 1 shows the dose-response curve of Stevia extract in the GlyT1 inhibition assay. Assay results are presented as the percentage inhibition of internalisation of radioactive glycine into the cells. FIG. 1 clearly demonstrates that Stevia extract can specifically inhibit the action of GlyT1 in a cellular assay.

FIG. 2a shows the error rate in the step-down test; a: significantly different from vehicle-treated, age-matched littermates during the training period. b: significantly different from vehicle-treated, age-matched littermates during the test period. c: significantly different from vehicle-treated, age-matched littermates during the washout period. d: significantly different from ginkgo biloba-treated mice during the washout period. In all cases, significance is denoted as p<0.05. These data show that Stevia-treated mice not only learned better than other groups but also retained their memory for a longer time period.

FIG. 2b shows results from the duration of latency step-down behavioral testing (to escape electric shock) b: significantly different from vehicle-treated, age-matched littermates during the test period. c: significantly different from vehicle-treated, age-matched littermates during the washout period. d: significantly different from Ginkgo biloba-treated mice during the washout period. In all cases, significance is denoted as p<0.05. Thus, mice treated with 150 mg/kg Stevia extract showed a significantly better learning and memory performance than their age-matched controls while, at the highest dose, Stevia-treated mice performed better than both the age-matched control mice and than the ginkgo biloba-treated positive controls.

FIG. 3a shows the latency period to locate the previous position of a hidden platform in the Morris water maze during training days. a: significantly different from vehicle-treated, age-matched littermates during the first day, emphasizing that treatment with a low dose of Stevia extract (50 mg/kg) significantly improved the learning performance of the animals. Significance is denoted as p<0.05.

FIG. 3b: Latency period to locate the previous position of a hidden platform in the Morris water maze on the testing day (Day 4). a: significantly different from vehicle-treated, age-matched littermates, showing that treatment with intermediate (150 mg/kg) and high (450 mg/kg) doses of Stevia extract significantly improved the memory performance of the animals, to the same extent as was observed in mice administered the reference substance, Ginkgo biloba and better than rolipram-treated animals. Significance is denoted as p<0.05.

FIG. 4a shows error rates in the shuttle box test: a: significantly different from vehicle-treated, age-matched littermates during the test period. b: significantly different from vehicle-treated, age-matched littermates during the extinction period. Significance is denoted as p<0.05. Thus, during the test period at the highest dose examined, Stevia-treated animals exhibited a significantly better memory performance than vehicle-treated littermates and an equivalent performance to mice administered the positive control compound, rolipram.

FIG. 4b shows escape times in the shuttle box test: a: significantly different from vehicle-treated, age-matched littermates during the test period. b: significantly different from Ginkgo biloba-treated, age-matched littermates during the test period. Significance is denoted as p<0.05. These results show that Stevia-treated mice (at doses of 150 and 450 mg/kg) learned to escape the foot-shock, their performance being better than that of both vehicle-treated control mice and mice administered the reference substance, Ginkgo biloba.

FIG. 5 shows visit duration in each corner 3 h before and after objects were presented. The arrows represent the placement of an object. Ginkgo biloba and Stevia extract significantly increased duration of visits to corners containing novel objects, compared with vehicle-treated control mice (p=0.004 and p=0.005, respectively). Both treatments thus increased the exploratory activity of mice in the object recognition test.

FIG. 6 shows the place error rate (percentage of visits to incorrect corners). Stevia extract treatment resulted in a significantly lower percent error rate compared with both vehicle-treated control mice and mice administered ginkgo biloba (p=0.012 and p=0.017, respectively). This effect was observed from the start of the module and was maintained throughout the first 12 h (active phase) and demonstrated that treatment with Stevia resulted in a higher level of attention and an improved memory performance.

FIG. 7 shows the side error rate (percentage of nosepokes at the incorrect side of the correct corner). Stevia extract again induced a significantly lower percent error rate compared with both vehicle-treated controls and Ginkgo-treated mice (p=0.002 and P=0.035, respectively. This effect was observed from the start of the module and was maintained throughout the first 12 h (active phase), indicating improved attention and memory in Stevia-treated mice.

### Stevia Extract

The Stevia extract may be made from any species of the genus Stevia, such as Stevia rebaudiana, Stevia eupatoria, Stevia ovata, Stevia plummerae, Stevia salicifolia and Stevia serrata. Generally, the Stevia extract should contain at least about 10-95% stevioside and steviol, preferably from about 40-85%.

As used throughout the specification and claims, the term “Stevia extract” is intended to be used broadly, and can encompass plant extracts made by conventional means, such as steam distillation, water-based extractions, alcohol-based extractions, or organic solvent-based extractions, such as ethyl acetate, and ion-exchange chromatography.

The only critical parameters regarding the Stevia extract are:
1) It should be acceptable for use in a nutraceutical for animal or human consumption. Thus the solvent to be employed for its preparation should be approved by the various regulatory agencies for the intended use. Therefore, preferred extracts are hot water, steam, alcohol and ethyl acetate.
2) It should contain sufficient amounts of stevioside, isosteviol and/or steviol to be efficacious.

Additionally, it has been found that dried plant parts (such as leaves) contain sufficient amounts of the active ingredients. As such, it is intended that dried forms of Stevia should also be included in this invention as valuable sources of Stevia extract and its constituents. Stevia extracts typically contain other compounds which may also be bioactive, and/or increase the bioavailability of the active components of Stevia. The amounts in which they are present in the Stevia extract will vary, based on a number of factors, including: the species of Stevia used, the growing conditions of the plant, and, of course, the processes used to prepare the Stevia extract. A typical Stevia extract prepared using aqueous extraction methods will contain steviol, isosteviol, stevioside and rebaudiosides.

### Stevia Extract and its Constituents Benefit Mental States

As previously described, the basis of memory, learning and mental stability is LTP, or the strengthening of neuronal connections, which occurs via activation of AMPA and NMDA receptors within the brain, particularly in the hippocampus. As glycine reuptake inhibitors, through their activity at GlyT1, Stevia extracts allow accumulation of glycine in the vicinity of the NMDA receptor, thus activating it and ultimately resulting in the induction of LTP, the main cellular mechanism involved in memory formation and consolidation.

Moreover, steviol, isosteviol and stevioside also induce activation of the same biochemical pathway (albeit at a different step than Stevia extract), leading to LTP induction, and are likewise beneficial in improving memory functions.

Stevia extract and its constituents can therefore activate hippocampal functions and improve memory formation and consolidation, as well as mental health. Thus, as used throughout the specification and claims, the term a “cognitive-function enhancing amount” means that the dosage is sufficient to activate hippocampal functions and can lead to improved cognitive function, which is explained further below.

Conditions Improved by this Invention:

In the context of this invention “treatment” also encompasses co-treatment as well as prevention, lessening the symptoms associated with a particular condition, decreasing the time of onset of a particular condition, lessening the severity of a condition, and decreasing the likihood that an asymptomatic individual will show a condition in the future.

Throughout this specification and claims, the term “improved cognitive function” is meant to refer to the conditions of supporting and maintaining cognitive wellness and balance, such as:
Enhanced learning, including:
   language processing
   problem solving
   intellectual functioning
Ability to cope with psychosocial burdens
Enhanced attention and concentration
Enhanced memory and the capacity for remembering, especially short-term memory
Enhanced mental alertness and mental vigilance, reduction of mental fatigue
Stabilisation of mental status including:
   Relieving post-partum conditions
   Relieving psychological burden due to separation of partners, children, death of beloved people or due to marital problems
   Relieving problems associated with change of domicile or work, or similar events
   Relieving stressful conditions following a traffic accident or other negative social pressure.
Stress relief, including:
   treatment, prevention and alleviation of symptoms related to work overload, exhaustion and/or burn out
   increased resistance or tolerance to stress
   favouring and facilitating relaxation in normal healthy individuals
“Condition improvement”, including:
   reducing irritability and tiredness
   Ability to cope with new situations
   reducing, preventing or alleviating physical and mental fatigue
   promoting good-quality sleep, that is to act against insomnia and sleep disorders and to increase energy in more general terms, in diseased or normal healthy individuals.

In a preferred aspect of the present invention the compositions may be used as nutritional supplements, particularly for people who may feel a need for enhanced cognitive function and/or psychosocial support. A non-exhaustive list of people who would benefit from enhanced cognitive function would include:
elderly people,
students or persons who are preparing for exams,
children who are engaged in a great deal of learning, i.e. infants, toddlers, pre-school children and school children,
construction workers, or those operating potentially dangerous machinery,
truck drivers, pilots, train drivers, or other transportation professionals,
air traffic controllers,
salespeople, executives, and other “high performance professionals”
police officers and military personnel,
housewives,
   or for anyone exposed to high amounts of stress in their daily work or who needs especially high attention/concentration/high mental and psychological performance in their daily work, such as those participating in sports, chess players, golfers, professional performers (actors, musicians and the like).

To achieve these improvements, administration over several days (for example at least six or ten days) is recommended, and administration daily for several weeks is generally preferred.

Aside from applications for humans, the compositions of this invention have additional uses in the veterinary world. Animals which can benefit from enhanced cognitive function include those animals which are subject to stressful conditions. Such conditions occur, for example, after capture or transport or may be due to housing conditions (such as change of domicile or owner), when the animals develop analogous disorders and are distressed or aggressive, or display stereotypic behaviour, or anxiety and obsessive-compulsive behaviour. Animals which are subject to stress would also include those which are racing animals (e.g. dogs, horses, camels), or used in various sports, performing animals (such as circus animals and those appearing on stage, television or in the movies) and horses which perform dressage and other highly disciplined routines.

Preferred “animals” are pets or companion animals and farm animals. Examples of pets are dogs, cats, birds, aquarium fish, guinea pigs, (jack) rabbits, hares and ferrets. Examples of farm animals are aquaculture fish, pigs, horses, ruminants (cattle, sheep and goats) and poultry.

### Nutraceutical Uses/Formulations/Dosages

The term “nutraceutical” as used herein denotes usefulness in both nutritional and pharmaceutical fields of application. Thus, novel nutraceutical compositions can be used as supplements to food and beverages and as pharmaceutical formulations for enteral or parenteral application which may be solid formulations, such as capsules or tablets, or liquid formulations, such as solutions or suspensions.

The nutraceutical compositions according to the present invention may further contain protective hydrocolloids (such as gums, proteins, modified starches), binders, film-forming agents, encapsulating agents/materials, wall/shell materials, matrix compounds, coatings, emulsifiers, surface active agents, solubilising agents (oils, fats, waxes, lecithins etc.), adsorbents, carriers, fillers, co-compounds, dispersing agents, wetting agents, processing aids (solvents), flowing agents, taste-masking agents, weighting agents, jellyfying agents, gel-forming agents, antioxidants and antimicrobials.

Moreover, a multi-vitamin and mineral supplement may be added to nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

The nutraceutical compositions according to the present invention may be in any galenic form that is suitable for administering to the body, especially in any form that is conventional for oral administration, e.g. in solid forms such as (additives/supplements for) food or feed, food or feed premix, fortified food or feed, tablets, pills, granules, dragées, capsules and effervescent formulations, such as powders and tablets, or in liquid forms, such as solutions, emulsions or suspensions as e.g. beverages, pastes and oily suspensions. The pastes may be incorporated in hard- or soft-shell capsules, whereby the capsules feature e.g. a matrix of (fish, swine, poultry, cow) gelatine, plant proteins or ligninsulfonate. Examples for other application forms are those for transdermal, parenteral or injectable administration. The dietary and pharmaceutical compositions may be in the form of controlled (delayed) release formulations.

Examples of food are dairy products including, for example, margarines, spreads, butter, cheese, yoghurts or milk-drinks.

Examples of fortified food are sweet corn, bread, cereal bars, bakery items, such as cakes and cookies, and potato chips or crisps.

Beverages encompass non-alcoholic and alcoholic drinks as well as liquid preparations to be added to drinking water and liquid food. Non-alcoholic drinks are e.g. soft drinks, sports drinks, fruit juices, lemonades, teas and milk-based drinks. Liquid foods are e.g. soups and dairy products. The nutraceutical composition containing Stevia extract, its constituents or an enriched Stevia extract may be added to a soft drink, an energy bar, or a candy, such that an adult consumes 1 to 1000 mg, more preferably 50-750 mg and most preferably 100-500 mg of Stevia extract, its constituents or an enriched Stevia extract per daily serving(s).

If the nutraceutical composition is a pharmaceutical formulation the composition further contains pharmaceutically acceptable excipients, diluents or adjuvants. Standard techniques may be used for their formulation, as e.g. disclosed in Remington's Pharmaceutical Sciences, 20th edition Williams & Wilkins, PA, USA. For oral administration, tablets and capsules are preferably used which contain a suitable binding agent, e.g. gelatine or polyvinyl pyrrolidone, a suitable filler, e.g. lactose or starch, a suitable lubricant, e.g. magnesium stearate, and optionally further additives. Preferred are formulations containing 10 to 750 mg, more preferably 50 to 500 mg, of the Stevia extract or its constituents, per administration unit, e.g. per tablet or capsule.

For animals including humans a suitable daily dosage of Stevia extract or its constituents, for the purposes of the present invention, may be within the range from 0.15 mg per kg body weight to about 10 mg per kg body weight per day. More preferred are nutraceutical compositions of the present invention which contain Stevia extract or its constituents, preferably in an amount sufficient to administer to a human adult (weighing about 70 kg) a dosage from about 10 mg/day to about 750 mg/day, preferably from about 50 mg/day to about 500 mg/day.

The following non-limiting Examples are presented to better illustrate the invention.

### Example 1

### Composition and Preparation of Stevia Extract

A typical Stevia aqueous extract disclosed by this invention contains:

| | |
|---|---|
| Stevioside | ca. 70% |
| Rebaudiosides | ca. 20% |
| Steviol, Isosteviol | ca. 10% |

The ground leaves of Stevia rebaudiana are mixed with hot water for 20-30 min. Subsequently, the aqueous extract is removed by draining, using pressure in order to achieve the maximum amount of extract. Several types of infusion/draining processes may be used. The extract is allowed to cool to room temperature. In order to remove particles, the extract may be allowed to rest while particulate matter settles out or may be centrifuged. The extract is subsequently dried, either by spray-drying or freeze-drying. This extract contains the sweetener principles, the plant pigments and other water-soluble components.

The Stevia extract (“Stevia leaves extract, 85% powder”; Catalogue number S1964) used in the following examples was purchased from Spectrum Laboratory Products Inc., Gardena, Calif., USA.

### Example 2

### Inhibition of Glycine Transporter 1 in a Cellular Assay

CHO cells stably expressing the human glycine transporter 1b cDNA (GlyT1) were routinely grown in Dulbecco's Modified Eagle's Medium (Invitrogen, Carlsbad, USA) containing 10% dialysed foetal calf serum, penicillin, streptomycin, proline and the antibiotic G418. Cells were harvested by trypsinisation one day prior to the assay and were seeded in the above mentioned medium. Immediately prior to the assay, the medium was replaced by uptake buffer containing 150 mM NaCl, 1 mM CaCl₂, 2.5 mM KCl, 2.5 mM MgCl₂, 10 mM Glucose and 10 mM N-2-hydroxyethylpiperazine-N′-2-ethanesulfonic acid (HEPES buffer).

Glycine uptake into the cells was determined by addition of 60 nM radio-labelled [³H]-glycine (Amersham Biosciences GE Healthcare, Slough, UK) and incubation for 30 minutes at room temperature. Following removal of unincorporated label by gentle washing three times with the above buffer, incorporated glycine was quantified by liquid scintillation counting.

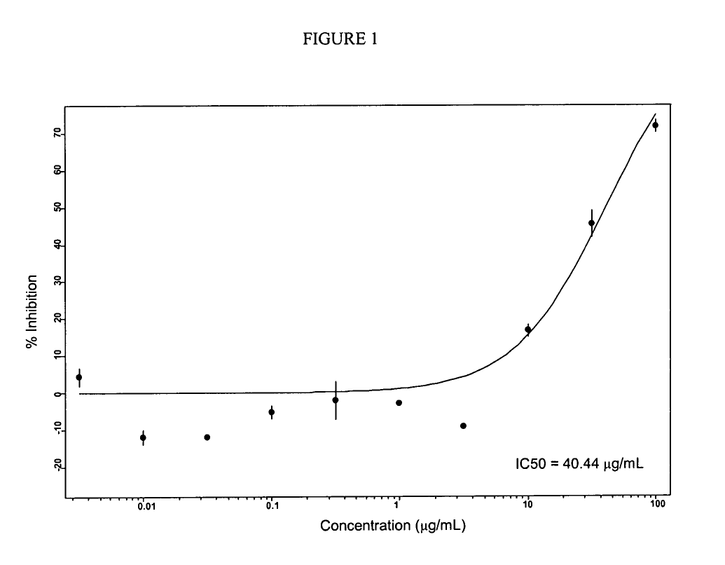

Glycine uptake via the GlyT1 transporter was inhibited by the addition of Stevia extract in a dose-dependent manner. Sarcosine, ORG24598 and ALX5407 (all from Sigma, St. Louis, USA) were used as known inhibitors of GlyT1. The measured IC₅₀ values for inhibition of glycine uptake (Stevia extract, extract constituents and reference compounds) and representative dose-response curve (Stevia extract) are shown in Table 2 and FIG. 1, respectively.

TABLE 2
Measured IC₅₀ values for inhibition of glycine uptake
into CHO cells by Stevia extract and its major components,
steviol, isosteviol and stevioside, in addition to the reference
compounds sarcosine, ORG24598 and ALX5407. Data is shown
as mean ± s.e.m. (where experiments were performed more than once);
IC₅₀ is stated as μg/ml for the extract and μM for pure compounds.

| | IC₅₀ for tritiated | |
|---|---|---|
| Substance | glycine uptake | |
| Stevia Extract (composition according to example 1) | 45 ± 6.9 | μg/ml |
| Steviol (Chromadex; Cat. No. ASB-00019352-025) | inactive | |
| Isosteviol (Wako Chemicals; Cat. no. 090-02341) | inactive | |
| Stevioside (Indofine Chemical Co.; Cat. no. 023718) | inactive | |
| Sarcosine (Sigma; Cat. no. S7672) | 35.9 | μM |
| ORG24598 (Sigma; Cat. no. O7639) | 0.02 | μM |
| ALX5407 (Sigma; Cat. no. A8977) | 6.46 | nM |

| | |
|---|---|
| Rebaudioside A | inactive |
| Gingko biloba (Huisong Pharmaceuticals, GB 102- | inactive |
| 070116) | |

### Example 3

### Hippocampal Slice Cultures

Seven-day-old Wistar rats were decapitated using a guillotine. In less than 1 minute the skull was opened, the cerebral hemispheres were separated and transferred and both hippocampi were dissected and transferred into ice-cold buffer containing 137 mM NaCl, 5 mM KCl, 0.85 mM Na₂HPO₄, 1.5 mM CaCl₂, 0.66 mM KH₂PO₄, 0.28 mM MgSO₄, 1 mM MgCl₂, 2.7 mM NaHCO₃, 1 mM Kynurenic acid and 0.6% D-glucose.

Transverse hippocampal slices (400 μm) were prepared using a vibrating blade microtome (VT1200S; Leica Microsystems (Schweiz) AG, Heerbrugg, Switzerland) in the same buffer. Hippocampal slices were individually placed on membrane inserts (Millicell Culture Plate Inserts, 0.4 μm) and cultivated at 35° C., 5% CO₂, 95% humidity in a medium containing a 1:1 mixture of BME and MEM (both from Invitrogen) containing 25% heat-inactivated horse serum, 1× GlutaMAX, 1× Penicillin/Streptomycin, 0.6% glucose and 1 mM Kynurenic acid (Stoppini 1991 J. Neurosci. Methods 37(2):173-82).

After 48 h in culture, synaptic NMDA receptors were activated by addition of Stevia extract, its major constituents or control substances for 15 min in 140 mM NaCl, 5 mM KCl, 1.3 mM CaCl₂, 25 mM HEPES (pH 7.3), 33 mM D-glucose and 0.02 mM bicuculline methiodide. Sarcosine (100 μM) and ALX5407 (20 nM) were used routinely as positive controls. An additional positive control comprised the addition of 200 μM glycine to sister cultures. After the treatments, sections were washed and fixed for immunohistochemistry. Markers of enhanced synaptic activity, normally associated with long-term potentiation, representing an ex vivo model of learning and memory were quantitated (see Table 3, below).

TABLE 3
Relative activation of synaptic markers after treatment with Stevia
extract or some of its constituent compounds (steviol, isosteviol,
stevioside and Rebaudioside A) in comparison with sister cultures treated
with buffer. The activation of any of these markers (or a combination
thereof) is observed in classical LTP experiments.

| Substance | pCREB | pMAPK | GluR1 |
|---|---|---|---|
| Stevia Extract | ± | + | 255% |
| Steviol | ++ | ± | 340% |
| Isosteviol | + | ± | 361% |
| Stevioside | ± | ++ | ± |
| Rebaudioside A | ± | ± | ± |
| Ginkgo biloba | ± | + | ± |

++++ shows a qualitative maximal activation,
++ and + signify a half-maximum and a moderate activation, respectively, while
± indicates no change in immunoreactivity;
for GluR1, values are shown as percentage increase of control values.

Treatment of hippocampal cultures with Stevia extract, steviol, isosteviol and stevioside induced one or more biochemical markers typical for LTP (pCREB: activated form of the cAMP response element binding protein; pMAPK: activated form of the mitogen-activated protein kinase; GluR1: cell surface presence of AMPA receptor 1).

### Example 4

### Effects of Stevia Extract in Three Traditional Rodent Models of Learning and Memory 1-Step-Down Test

Mice were subjected to an associative learning and memory paradigm, the step-down test. Mice were individually placed in a reaction box, the floor of which was fitted with a 36V electric grid. When animals receive an electric shock, their normal reaction is to jump up onto an insulated platform to avoid the pain stimulus. Thus, the majority of animals that step down on to the grid, would, upon receiving the electric shock, rapidly jump back up on to the platform. On Day 0, animals were trained for 5 min, and the number of times each mouse was shocked, i.e. made an error, was noted. This data constituted the learning data. Mice were re-tested 24 h (Day 1, training day) and 48 h (Day 2, test day) later, with these trials serving as the memory tests. The number of animals shocked in each group, the time prior to first stepping down from the platform (latency) and the number of errors in the first 3 min were recorded. Six days after conclusion of training, on Day 8, memory decay was tested (washout test).

The study included six test groups (n=12 per group). All mice were administered test substances or vehicle via daily oral gavage (10 ml/kg) for 30 days prior to training the animals. Stevia extract was tested at 3 doses, 50 (low), 150 (intermediate) and 450 (high) mg/kg, while the reference substance, Ginkgo biloba, was administered at a dose of 100 mg/kg. The pharmacological positive control, rolipram (0.1 mg/kg), was administered by intraperitoneal injection 30 min before testing.

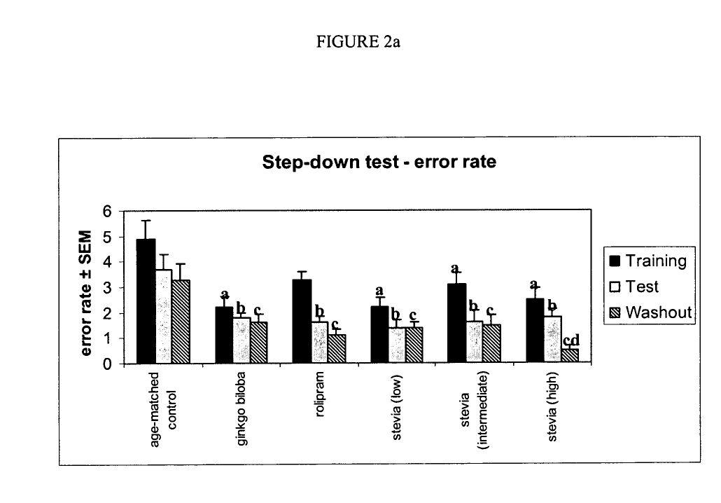

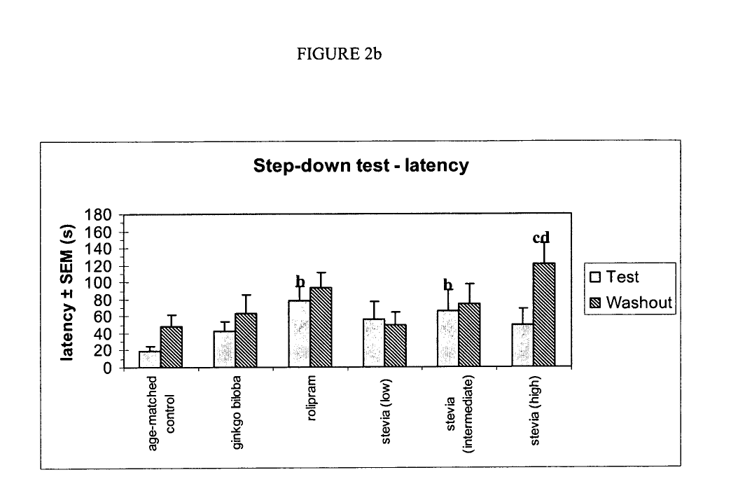

When compared to vehicle-treated littermates (negative control), all groups of Stevia-treated animals exhibited a significantly better learning and memory performance during the training and memory phase and after the wash-out period, as shown by reductions in error rates (FIG. 2a). Furthermore, during the training phase Stevia-treated mice (at 50 mg/kg) performed as well as mice administered the reference compound, ginkgo biloba, and better than rolipram-treated mice; during the test phase all three doses of Stevia extract resulted in similar performance to that observed in both ginkgo- and rolipram-treated mice; during the washout phase, mice administered low and intermediate doses of Stevia extract again performed as well as mice treated with the reference substance and positive control, while the highest dose of Stevia induced a significantly better performance than was seen in ginkgo-treated mice.

During the test phase, the latency period was significantly increased in mice administered the intermediate dose of Stevia extract, when compared with vehicle-treated control mice. Furthermore, this effect was equivalent to that observed in rolipram-treated mice. During the washout phase, the highest dose of Stevia extract induced a significant increase in latency, compared with both vehicle-treated control mice and mice administered ginkgo biloba extract (FIG. 2b).

Thus, to summarize, Stevia extract enabled improved learning and memory performance, to a similar, or better, extent as a natural reference substance, ginkgo biloba, and a pharmaceutical positive control compound, rolipram.

FIG. 2a: Error rate in the step-down test; a: significantly different from vehicle-treated, age-matched littermates during the training period. b: significantly different from vehicle-treated, age-matched littermates during the test period. c: significantly different from vehicle-treated, age-matched littermates during the washout period. d: significantly different from ginkgo biloba-treated mice during the washout period. In all cases, significance is denoted as p<0.05. These data show that Stevia-treated mice not only learned better than other groups but also retained their memory for a longer time period.

FIG. 2b shows results for Step-down behavioral testing; duration of latency to escape electric shock b: significantly different from vehicle-treated, age-matched littermates during the test period. c: significantly different from vehicle-treated, age-matched littermates during the washout period. d: significantly different from ginkgo biloba-treated mice during the washout period. In all cases, significance is denoted as p<0.05. Thus, mice treated with 150 mg/kg Stevia extract showed a significantly better learning and memory performance than their age-matched controls while, at the highest dose, Stevia-treated mice performed better than both the age-matched control mice and than the ginkgo biloba-treated positive controls.

### Example 5

### Effects of Stevia Extract in Three Traditional Rodent Models of Learning and Memory 2-Morris Water Maze

The Morris water maze is one of the best accepted paradigms to test spatial memory in rodents. Mice have to swim in a round pool and to search for a hidden platform, aided by the use of visual cues in the experimentation room.

The same doses and treatment time were employed here as for the step-down test paradigm.

The Morris water maze (diameter=1.5 m) used in this study included a hidden platform (10 cm×10 cm), located 10 cm from the edge of the pool in the northwest quadrant (at “10 o'clock”). On Day 0, mice were acclimatized to the testing arena and received two training trials, during which they were trained to swim to the platform.

On Days 1 to 3 the platform was removed from the water maze and mice received two training trials per day. After each trial (90 sec), the platform was put back at the same location to allow the mice to find it and to climb onto it. This procedure ensured that mice efficiently learned the position of the platform on each training day and recalled the platform's position in subsequent trials. The initial time to locate the area where the platform had been located (First Cross Platform Time; FCPT) and the number of times the mice swam across the area where the platform had been located (Crossing Times) were recorded and provided an average score for each training day (Days 1 to 3: Training Scores). On Day 4, mice received a further two trials, the results of which were the Testing Scores.

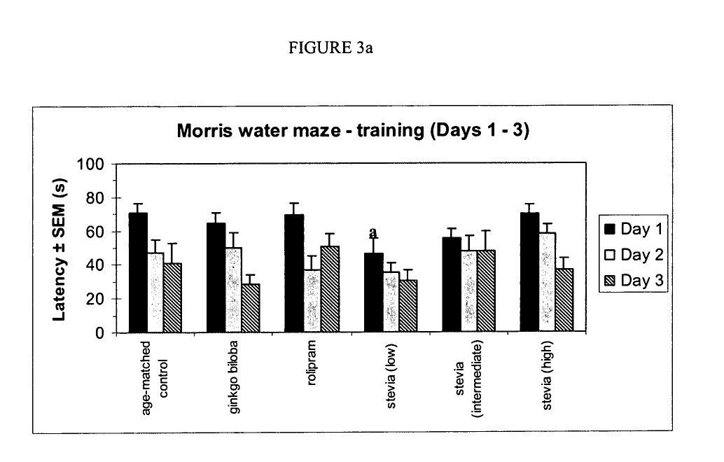

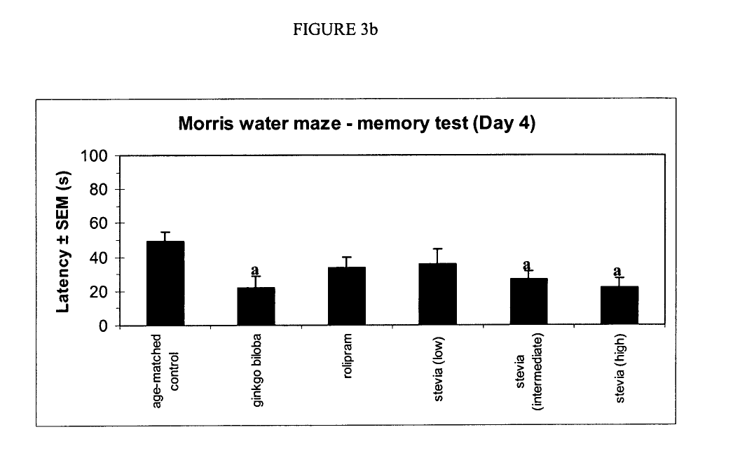

On Day 1 of the training sessions, mice administered the lowest dose of Stevia performed significantly better than vehicle-treated control mice and than mice administered the positive control compound, rolipram (FIG. 3a). On day 4, during the test trial, mice treated with the intermediate and high doses of Stevia showed a significantly shorter latency time compared to the age-matched control animals (FIG. 3b). Thus, these data indicate that treatment with Stevia extract affects both learning and memory performance of mice, and that this effect is dependent upon the dose utilized.

FIG. 3a shows the latency period to locate the previous position of a hidden platform in the Morris water maze during training days. a: significantly different from vehicle-treated, age-matched littermates during the first day, emphasizing that treatment with a low dose of Stevia extract (50 mg/kg) significantly improved the learning performance of the animals. Significance is denoted as p<0.05.

FIG. 3b shows the latency period to locate the previous position of a hidden platform in the Morris water maze on the testing day (Day 4). a: significantly different from vehicle-treated, age-matched littermates, showing that treatment with intermediate (150 mg/kg) and high (450 mg/kg) doses of Stevia extract significantly improved the memory performance of the animals, to the same extent as was observed in mice administered the reference substance, ginkgo biloba and better than rolipram-treated animals. Significance is denoted as p<0.05.

### Example 6

### Effects of Stevia Extract in Three Traditional Rodent Models of Learning and Memory

### 3-Shuttle Box Test

The paradigm underlying this animal test is active avoidance of adverse stimuli. One part of the shuttle box is equipped with a grid that delivers electric shocks. An auditory signal (a beeping noise, lasting 7 s) precedes the electric shock. The animal has to learn to escape to the other side of the shuttle box thereby crossing an infrared light beam.

The same doses and treatment time were employed here as for the step-down test paradigm.

The mice were trained once per day, for 5 days. The training program constituted 15 sessions; each session included (1) a 15 s pause, (2) a 7 s auditory signal (“beeping period”) and (3) an 8 s electric shock (“stimulating period”). If a mouse crossed one of the two infrared light beams at either end of the shuttle box during the beeping or stimulating period, it progressed to the next session. The active escape times (beeping period) and passive escape times (stimulating period) were recorded and the total escape times were calculated. If a mouse did not cross an infrared light beam during the beeping period it was recorded as an error. The errors and escape times recorded on Day 4 were taken as the training scores, on Day 5 as the test scores and on Day 8 as extinction scores.

During the test phase (Day 5), the highest dose of Stevia extract induced significantly fewer errors, compared with vehicle-treated control mice; the effect on error rate was equivalent to that observed in rolipram-treated positive control mice. In addition, the intermediate and high doses of Stevia extract significantly reduced escape time, compared with both vehicle-treated and ginkgo-treated mice. Thus, these data again indicate that treatment with Stevia extract affects learning performance of mice, but that this effect is dependent upon the dose utilized.

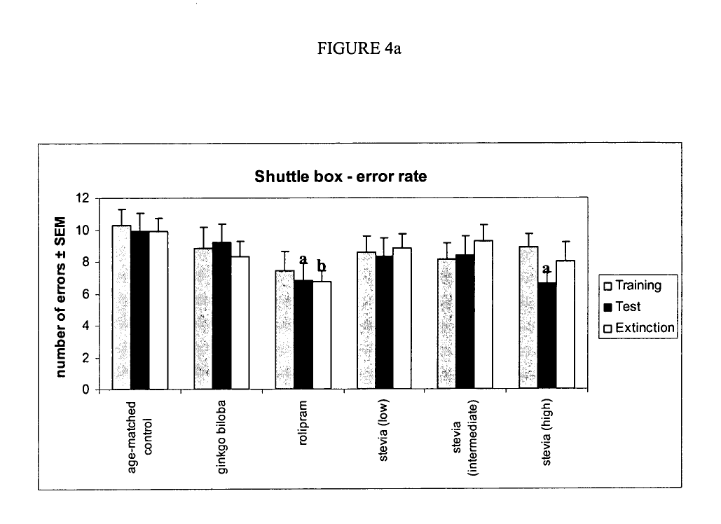

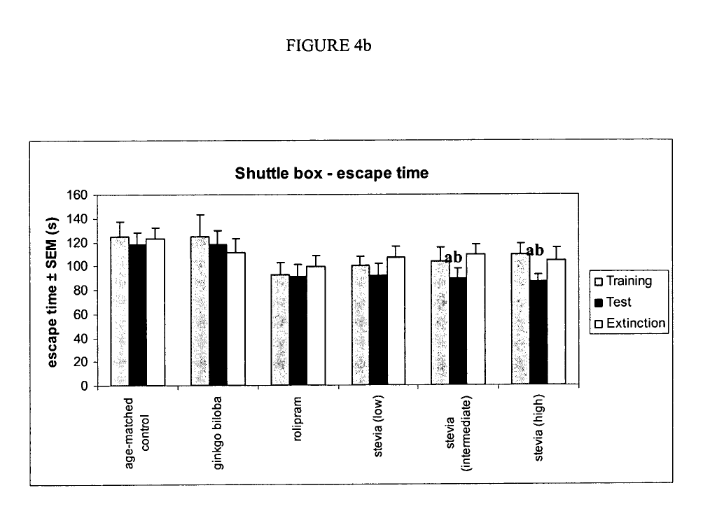

FIG. 4a shows the error rates in the shuttle box test: a: significantly different from vehicle-treated, age-matched littermates during the test period. b: significantly different from vehicle-treated, age-matched littermates during the extinction period. Significance is denoted as p<0.05. Thus, during the test period at the highest dose examined, Stevia-treated animals exhibited a significantly better memory performance than vehicle-treated littermates and an equivalent performance to mice administered the positive control compound, rolipram.

FIG. 4b shows escape times in the shuttle box test: a: significantly different from vehicle-treated, age-matched littermates during the test period. b: significantly different from Ginkgo biloba-treated, age-matched littermates during the test period. Significance is denoted as p<0.05. These results show that Stevia-treated mice (at doses of 150 and 450 mg/kg) learned to escape the foot-shock, their performance being better than that of both vehicle-treated control mice and mice administered the reference substance, Ginkgo biloba.

### Example 7

### Effects of Stevia Extract in a New, Totally Automated, Rodent Model of Learning and Memory

The cognitive performance of mice treated with Stevia extract or Ginkgo biloba was compared with that of vehicle-treated, age-matched controls in the IntelliCage®, a system which enables automated monitoring of spontaneous and learning behavior of mice in a homecage-like environment (NewBehavior AG, Zürich, Switzerland, www.newbehavior.com; Galsworthy et al. 2005, Behav Brain Res 157: 211-217; Onishchenko et al. 2007, Toxicol Sci 97: 428-437). Individual mice are recognized by sensors within the IntelliCage corners reading a transponder (reference identification tag) which is implanted into the scruff of the mouse's neck. Each IntelliCage® is essentially a large cage (37.5×55×20.5 cm), into which is placed a metal frame, comprising four recording (operant) chambers. The recording chambers fit into the corners of the cage, each covering a 15×15×21 cm right-angled triangular area of floor space. In-cage antennae enable automatic monitoring of each individual mouse's corner visits; photo-beams within each corner enable automated recording of individual nosepokes and licks of the water bottle spouts. Four triangular mouse shelters are placed in the center of the cage, above which is situated a food hopper, enabling ad libitum access to food.

Each recording chamber comprises: (1) a plastic ring (30 mm inner diameter) which serves as an entrance into the chamber and houses the circular antenna which registers corner visits; (2) a grid floor, which the mice sit on once they have entered the chamber; (3) two circular openings (13 mm diameter) which enable access to water bottle spouts; each opening is crossed by photo-beams which register nose-pokes; (4) two motorized doors, which allow (door open) or prohibit (door closed) access to the water bottle spouts; (5) two water bottles; (6) tubing, through which air-puffs can be delivered as aversive stimulation; (7) different colored light diodes, which can be used for conditioning experiments.

### Experimental Phase:

The study included three test groups (n=12-14 per group): vehicle (control), Ginkgo biloba (100 mg/kg) and Stevia extract (450 mg/kg). All mice were administered test substances or vehicle via daily oral gavage (10 ml/kg) throughout the 8 week study.

During an initial adaptation period (4-5 days) mice had free access to all corners, water and feed and could freely explore the cage. Subsequently, mice had to learn to apply nose-pokes (nose-poke adaptation module, 3 days); all doors were initially closed (access to water was prohibited) and mice had to perform a nose-poke in order to open a door and to reach a water bottle spout. Data collected comprised several parameters, such as the least-preferred corner of each individual mouse, which was noted for programming the next modules.

### Object Recognition

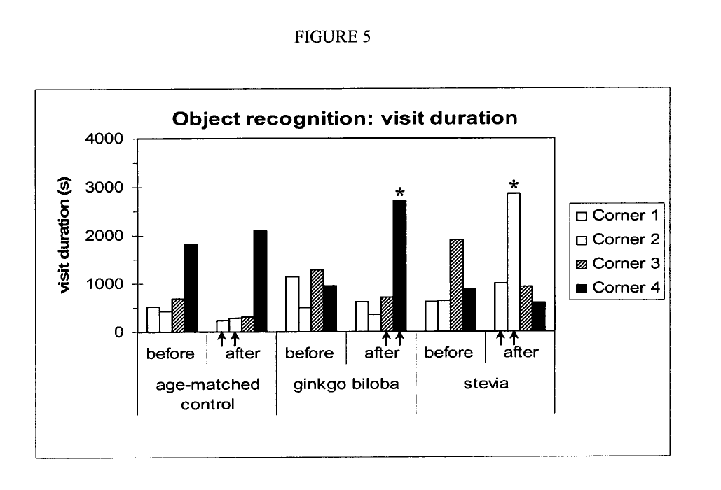

To test the intrinsic exploratory activity of the mice, two identical objects were placed in corners 1 and 2 or in corners 3 and 4, which included the least-preferred corner of each group, respectively. The animals had the opportunity to freely explore the cage, with full access to water and feed. Visits were recorded 3 h before and 3 h after the objects were presented. The visiting pattern of the control group did not change after presentation of the objects in Corners 1 and 2, while both ginkgo and Stevia treatment resulted in significant increases in duration of visits to corners containing novel objects (Corners 4 and 2, respectively) (FIG. 5). Thus, chronic administration of Stevia extract significantly increased exploratory activity of mice, to the same extent as Ginkgo biloba.

FIG. 5 shows the visit duration in each corner 3 h before and after objects were presented. The arrows represent the placement of an object. Ginkgo biloba and Stevia extract significantly increased duration of visits to corners containing novel objects, compared with vehicle-treated control mice (p=0.004 and p=0.005, respectively). Both treatments thus increased the exploratory activity of mice in the object recognition test.

### Side Discrimination

This module was designed to test attention and associative memory. One correct corner was assigned to each mouse. In this corner only one side (of two) was assigned as correct, and was indicated to the animals by a green LED. At the correct side animals could make a nosepoke and subsequently drink from the water bottle. During this module the place errors (i.e. percentage of visits to incorrect corners) and side errors (i.e. percentage of nosepokes at the incorrect side of the correct corner) were recorded.

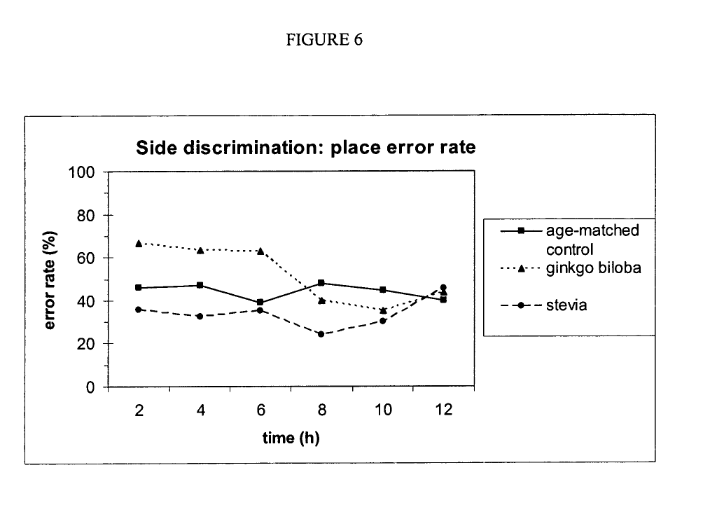

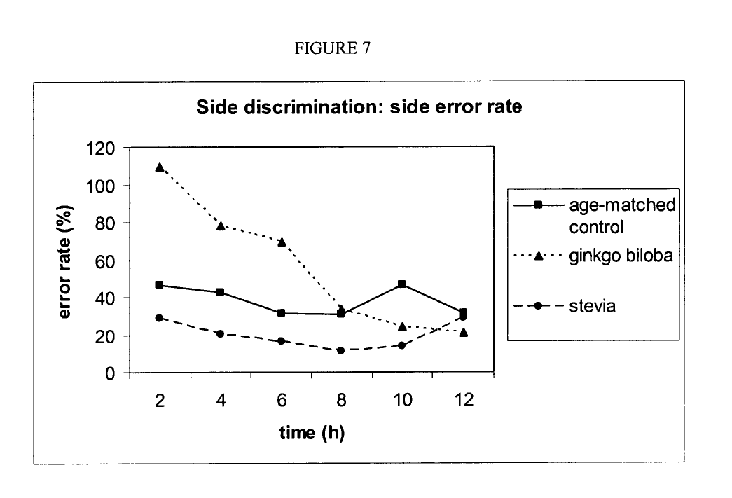

These data indicate improved attention following chronic treatment with Stevia extract, since both the place error rate (FIG. 6) and side error rate (FIG. 7) were significantly lower than those of both vehicle- and Ginkgo-treated mice throughout the first 12 h (active phase) of the test.

FIG. 6 shows the place error rate (percentage of visits to incorrect corners). Stevia extract treatment resulted in a significantly lower percent error rate compared with both vehicle-treated control mice and mice administered Ginkgo biloba (p=0.012 and p=0.017, respectively). This effect was observed from the start of the module and was maintained throughout the first 12 h (active phase) and demonstrated that treatment with Stevia resulted in a higher level of attention and an improved memory performance.

FIG. 7 shows the side error rate (percentage of nosepokes at the incorrect side of the correct corner). Stevia extract again induced a significantly lower percent error rate compared with both vehicle-treated controls and Ginkgo-treated mice (p=0.002 and p=0.035, respectively). This effect was observed from the start of the module and was maintained throughout the first 12 h (active phase), indicating improved attention and memory in Stevia-treated mice.

In summary, results from the automated IntelliCage® studies confirm the behavioural results from classical tests (Examples 4, 5 and 6) that treatment with Stevia extract for 8 weeks results in a significant improvement of learning and memory in mice when compared to vehicle-treated littermates. In some cases, the improvement in performance seen in Stevia-treated mice was even greater than that observed in mice administered either the natural reference substance, Ginkgo biloba, or the pharmaceutical positive control compound, rolipram.

### Example 8

### Preparation of a Soft Gelatine Capsule

A soft gelatine capsule may be prepared comprising the following ingredients:

| Ingredient | Amount per Capsule |
|---|---|
| Stevia extract (freeze- | 200 mg |
| dried/spray-dried) | |
| Lecithin | 50 mg |
| Soy bean oil | 250 mg |

Two capsules per day for 3 months may be administered to a human adult. Cognitive function, alertness and the ability to focus on work are seen to improve.

### Example 9

### Preparation of an Instant Flavoured Soft Drink

| Ingredient | Amount [g] |
|---|---|
| Stevia extract | 0.5 |
| Sucrose, fine powder | 763.1 |
| Ascorbic acid, fine powder | 2.0 |
| Citric acid anhydrous powder | 55.0 |
| Lemon flavour | 8.0 |
| Trisodium citrate anhydrous powder | 6.0 |
| Tricalciumphosphate | 5.0 |
| β-Carotene 1% CWS from DNP AG, | 0.4 |
| Kaiseraugst, Switzerland | |
| Total amount | 840 |

All ingredients are blended and sieved through a 500 μm sieve. The resulting powder is put in an appropriate container and mixed in a tubular blender for at least 20 minutes. For preparing the drink, 105 g of the obtained mixed powder are mixed with sufficient water to produce one litre of beverage.

The ready-to-drink soft drink contains ca. 15 mg enriched Stevia extract per serving (250 ml). As a strengthener and for general well-being 2 servings per day (500 ml) may be drunk. Cognitive function, alertness and the ability to focus on work are seen to improve.

### Example 10

### Preparation of a Fortified Non-Baked Cereal Bar

| Ingredient | Amount [g] |
|---|---|
| Stevia extract | 0.3 |
| Water | 54.0 |
| Salt | 1.5 |
| Glucose syrup | 130.0 |
| Invert sugar syrup | 95.0 |
| Sorbitol Syrup | 35.0 |
| Palm kernel fat | 60.0 |
| Baking fat | 40.0 |
| Lecithin | 1.7 |
| Hardened palm-oil | 2.5 |
| Dried and cut apple | 63.0 |
| Cornflakes | 100.0 |
| Rice crispies | 120.0 |
| Wheat crispies | 90.0 |
| Roasted hazelnut | 40.0 |
| Skimmed milk powder | 45.0 |
| Apple flavour 74863-33 | 2.0 |
| Citric acid | 5.0 |
| Total amount | 885 |

The enriched Stevia extract is premixed with skimmed milk powder and placed in a planetary bowl mixer. Cornflakes and rice crispies are added and the total is mixed gently. Then the dried and cut apples are added. In one cooking pot, water and salt are mixed in the amounts given above (solution 1). In a second cooking pot, glucose-, invert sugar- and sorbitol-syrups are mixed in the amounts given above (solution 2). The fat phase constitutes a mixture of baking fat, palm kernel fat, lecithin and emulsifier. Solution 1 is heated to 110° C. Solution 2 is heated to 113° C. and then cooled in a cold water bath. Subsequently, solutions 1 and 2 are combined. The fat phase is melted at 75° C. in a water bath, then added to the combined mixture of solutions 1 and 2. Apple flavour and citric acid are added to the liquid sugar/fat mix. The liquid mass is added to the dry ingredients and mixed well in the planetary bowl mixer. The mass is put on a marble plate and rolled to the desired thickness. The mass is cooled down to room temperature and cut into pieces. The non-baked cereal bar contains ca. 10 mg enriched Stevia extract per serving (30 g). For general well-being and energising 1-2 cereal bars may be eaten per day. Cognitive function, alertness and the ability to focus on work are seen to improve.

### Example 11

### Dry Dog Feed Containing Stevia Extract

A commercial basal diet for dogs (e.g. Mera Dog “Brocken”, MERA-Tiernahrung GmbH, Marienstraβe 80-84, D-47625 Kevelaer-Wetten, Germany) is sprayed with a solution of Stevia extract in water, together with antioxidants such as vitamin C (e.g. ROVIMIX® C-EC from DSM Nutritional Products Ltd, Kaiseraugst, Switzerland) and its derivatives, i.e. sodium ascorbyl monophosphate (e.g. STAY-C® 50 from DSM Nutritional Products Ltd, Kaiseraugst, Switzerland) or a mixture of tri-, di- and mono-phosphate esters of sodium/calcium L-ascorbate (e.g. ROVIMIX® STAY-C® 35 from DSM Nutritional Products Ltd, Kaiseraugst, Switzerland) in an amount sufficient to administer to a dog a daily dose of 4 mg Stevia extract per kg body weight. The food composition is dried to contain dry matter of about 90% by weight. For an average dog of 10 kg body weight to consume approx. 200 g dry feed per day, the dog food contains approx. 200 mg Stevia extract per kg food. For heavier dogs, the feed mix is prepared accordingly. For reduction of stress, fear and aggressiveness in dogs, the food can be given to dogs in animal shelter farms on a regular basis. Before veterinarian visits or stays in veterinarian clinics or holiday separation, the food is given at least one week before, during the stressful event and one week thereafter.

### Example 12

### Wet Cat Food Containing Stevia Extract

A commercial basal diet for cats (e.g. Happy Cat “Adult”, Tierfeinnahrung, Südliche Hauptstraβe 38, D-86517 Wehringen, Germany) is mixed with a solution of Stevia extract in water, together with antioxidants such as vitamin C (e.g. ROVIMIX® C-EC from DSM Nutritional Products Ltd, Kaiseraugst, Switzerland) and its derivatives, i.e. sodium ascorbyl monophosphate (e.g. STAY-C® 50 from DSM Nutritional Products Ltd, Kaiseraugst, Switzerland) or a mixture of tri-, di- and mono-phosphate esters of sodium/calcium L-ascorbate (e.g. ROVIMIX® STAY-C® 35 from DSM Nutritional Products Ltd, Kaiseraugst, Switzerland) in an amount sufficient to administer to a cat a daily dose of 4 mg Stevia extract per kg body weight. For an average cat of 5 kg of body weight to consume approx. 400 g of wet food, the cat food contains 50 mg Stevia extract per kg food. The food composition is dried to contain dry matter of about 90% by weight. For reduction of stress, fear and aggressiveness in cats, the food can be given to cats in animal shelter farms on a regular basis. Before veterinarian visits or stays in veterinarian clinics, the food is given at least one week before, during the stressful event, and one week thereafter.

### Example 13

### Dog Treats Containing Stevia Extract

Commercial dog treats (e.g. Mera Dog “Biscuit” for dogs as supplied by Mera Tiernahrung GmbH, Marienstrasse 80-84, 47625 Kevelaer-Wetten, Germany) are sprayed with a solution of Stevia extract in water, together with antioxidants such as vitamin C (e.g. ROVIMIX® C-EC from DSM Nutritional Products Ltd, Kaiseraugst, Switzerland) and its derivatives, i.e. sodium ascorbyl monophosphate (e.g. STAY-C® 50 from DSM Nutritional Products Ltd, Kaiseraugst, Switzerland) or a mixture of tri-, di- and mono-phosphate esters of sodium/calcium L-ascorbate (e.g. ROVIMIX® STAY-C® 35 from DSM Nutritional Products Ltd, Kaiseraugst, Switzerland) in an amount sufficient to administer to the treats 0.5-5 mg Stevia extract per g treats. The food composition is dried to contain dry matter of about 90% by weight. To reduce fear and tension, the treat can be given during the day in addition to the food, or when feeding is not warranted, i.e. while travelling, for up to 5 times per day.

### Example 14

### Cat Treats Containing Stevia Extract

Commercial cat treats (e.g. Whiskas Dentabits for cats as supplied by Whiskas, Masterfoods GmbH, Eitzer Str. 215, 27283 Verden/Aller, Germany) are sprayed with a solution of Stevia extract in water, together with antioxidants such as vitamin C (e.g. ROVIMIX® C-EC from DSM Nutritional Products Ltd, Kaiseraugst, Switzerland) and its derivatives, i.e. sodium ascorbyl monophosphate (e.g. STAY-C® 50 from DSM Nutritional Products Ltd, Kaiseraugst, Switzerland) or a mixture of tri-, di- and mono-phosphate esters of sodium/calcium L-ascorbate (e.g. ROVIMIX® STAY-C® 35 from DSM Nutritional Products Ltd, Kaiseraugst, Switzerland) in an amount sufficient to administer to the treats 0.5-5 mg Stevia extract per g treats. The food composition is dried to contain dry matter of about 90% by weight. To reduce fear and tension, the treat can be given during the day in addition to the food, or when feeding is not warranted, i.e. while travelling, for up to 5 times per day.

1. A method of improving cognitive function and/or psychological wellbeing in an animal or human comprising administering a cognitive function-improving amount of a compound selected from the group consisting of:
steviol, steviolbioside, stevioside, rebaudioside A, rebaudioside B, rebaudioside C (dulcoside B), rebaudioside D, rebaudioside E, rebaudioside F, rubusoside, dulcoside A, isosteviol, and mixtures thereof.

2. A method according to claim 1 wherein the compound is present in a Stevia extract.

3. A method according to claim 2 where in the Stevia extract contains at least about 10-90% stevioside, steviol and/or isosteviol.

4. A method according to claim 1 wherein the cognitive function and/or psychological wellbeing is selected from the group consisting of: maintaining cognitive wellness and balance, learning, language processing, problem solving, intellectual functioning, ability to cope with psychosocial burdens, attention and concentration, memory, the capacity for remembering, mental alertness, mental vigilance, mental fatigue, stabilisation of mental status, a stress reliever, work-overload stress, stress-related exhaustion and/or burn out, and to promote relaxation.

5. A composition used in the manufacture of a nutraceutical or medicament for improving cognitive function in an animal or human, which comprises a cognitive function-improving amount of a compound selected from the group consisting of: steviol, steviolbioside, stevioside, rebaudioside A, rebaudioside B, rebaudioside C (dulcoside B), rebaudioside D, rebaudioside E, rebaudioside F, rubusoside, dulcoside A, isosteviol, and mixtures thereof.

6. A composition according to claim 5 wherein the compound is present in a Stevia extract.

7. A composition according to claim 6 wherein the Stevia extract contains at least about 10-90% stevioside, steviol and/or isosteviol.

8. A composition according to claim 5 wherein the cognitive function is selected from the group consisting of: maintaining cognitive wellness and balance, learning, language processing, problem solving, intellectual functioning, ability to cope with psychosocial burdens, attention and concentration, memory, the capacity for remembering, mental alertness, mental vigilance, mental fatigue, stabilisation of mental status, a stress reliever, work overload stress, stress-related exhaustion and/or burn out, and to promote relaxation.

9. A nutraceutical comprising a cognitive function-improving amount of a compound selected from the group consisting of:
steviol, steviolbioside, stevioside, rebaudioside A, rebaudioside B, rebaudioside C (dulcoside B), rebaudioside D, rebaudioside E, rebaudioside F, rubusoside, dulcoside A, isosteviol, and mixtures thereof.

10. A nutraceutical according to claim 9 which is for veterinary use.

11. A nutraceutical according to claim 9 wherein the cognitive function is selected from the group consisting of: maintaining cognitive wellness and balance, learning, language processing, problem solving, intellectual functioning, ability to cope with psychosocial burdens, attention and concentration, memory, the capacity for remembering, mental alertness, mental vigilance, mental fatigue, stabilisation of mental status, a stress reliever, work overload stress, stress-related exhaustion and/or burn out, and to promote relaxation.
